(19) United States
(12) Patent Application Publication
(10) Pub. No.: US 2018/0364233 A1
(43) Pub. Date: Dec. 20, 2018

(54) METHODS AND PHARMACEUTICAL COMPOSITIONS FOR THE TREATMENT OF SYSTEMIC SCLEROSIS

(71) Applicants: INSERM (INSTITUT NATIONAL DE LA SANTÉ ET DE LA RECHERCHE MÉDICALE), Paris (FR); UNIVERSITÉ D'AIX MARSEILLE, Marseille Cedex 07 (FR); CENTRE NATIONAL DE LA RECHERCHE SCIENTIFIQUE (CNRS), Paris (FR); UNIVERSITÉ PARIS DESCARTES, Paris (FR); ASSISTANCE PUBLIQUE-HÔPITAUX DE PARIS (APHP), Paris (FR)

(72) Inventors: Philippe NAQUET, Marseille Cedex 09 (FR); Frédéric BATTEUX, Paris (FR); Niloufar KAVIAN, Paris (FR)

(21) Appl. No.: 15/781,839

(22) PCT Filed: Dec. 7, 2016
(86) PCT No.: PCT/EP2016/080095

§ 371 Date: Jun. 6, 2018

(30) Foreign Application Priority Data
Dec. 8, 2015 (EP) 15306955.4

(51) Int. Cl. G01N 33/573

(52) U.S. Cl. CPC G01N 33/573; G01N 2800/24; G01N 2333/98

(57) ABSTRACT

The present invention relates to methods and pharmaceutical compositions for the treatment of systemic sclerosis. In particular, the present invention relates to a method of treating systemic sclerosis in a subject in need thereof comprising administering to the subject a therapeutically effective amount of vanin-1 inhibitor.

### FIELD OF THE INVENTION

The present invention relates to methods and pharmaceutical compositions for the treatment of systemic sclerosis.

### BACKGROUND OF THE INVENTION

Systemic sclerosis (SSc) is an auto-immune disorder characterized by vascular alterations and fibrosis of the skin and visceral organs, that results from dysfunctions in endothelial, fibroblastic and immunocompetent cells (1-2). The pathophysiology of the disease remains poorly understood and no curative therapy is available. Several reports have emphasized the role of oxidative and inflammatory stresses as factors triggering the activation of fibroblasts and endothelial cells, and the alteration of self-antigens leading to the breach of tolerance (3-6).

Located at the crossroads of inflammation, oxidative stress, fibrosis and endothelial alterations the vanin/pantetheinase pathway could play a role in SSc pathogenesis. The vanin family genes encode pantetheinase enzymes and vanin-1 is the predominant iso form of vanins in humans and mice (7-9). Pantetheinase metabolizes pantetheine (the reduced form of pantethine) and generates cysteamine and pantothenic acid (vitamin B5) (10). Pantethine, the substrate of vanin-1, is endowed with cytoprotective properties towards endothelial cells (EC) that can improve the vasculopathy observed in several inflammatory conditions. The administration of pantethine prevents cerebral malaria in mice infected with Plasmodium berghei by preserving the blood brain barrier (11). The molecule also exerts beneficial effects on peripheral vasculature in patients with various types of hyperlipoproteinemia (12-14). Therefore, hyperactivity of the vanin-1/pantetheinase pathway may reduce the vasculoprotective effects of pantethine. By hydrolysis of pantethine, vanin-1 delivers cysteamine and pantothenic acid to tissues. Upon pro-oxidant conditions, cysteamine can be converted into cystamine that can regulate the function of other target proteins by a disulfide reaction (15, 16). In particular, cystamine reduces glutathione production by inhibiting γ-glutamyl synthase, the rate limiting enzyme of glutathione synthesis, thus strongly reducing ROS (reactive oxygen species) scavenging. On the other hand, pantothenic acid, the other by-product of vanin-1, exerts profibrotic effects by promoting the proliferation and migration of dermal fibroblasts, and collagen synthesis (17) (18, 19). Studies using vanin-1 knock-out mice (vnn1−/− mice) have confirmed the involvement of this enzyme in several inflammatory conditions (15, 20-22). For instance, vnn1−/− mice control better than wild-type mice inflammatory reactions in response to Schistosoma- and Coxiella burnettii-induced infections (15, 22). Also, vnn-1 deficient mice resist to experimental inflammatory colitis better than wild-type mice (23). Thus, the proinflammatory effects of vanin-1 are at least partly related to the role of vanin-1 as an oxidative stress sensor regulating glutathione (GSH) levels and redox homeostasis. If vanin-1 induces ROS through the depletion of GSH, ROS play also a key role in the regulation of vanin-1 expression. Indeed, vanin-1 is upregulated in tissues exposed to oxidative stress through the activation of the Vnn1 gene promoter that contains ARE (antioxidant response element) and PPRE (PPAR responsive element) (24, 25). Therefore, once ROS induce vanin-1 expression, an amplification loop may occur to favour further inflammation, fibrosis and vasculopathy. Moreover, as vanin-1 favours ROS production, this pathway may participate in the auto-immune process in SSc through the oxidation of DNA Topoisomerase-1, triggering the breach of immune tolerance and the production of auto-antibodies as previously described (3, 26-28).

### SUMMARY OF THE INVENTION

The present invention relates to methods and pharmaceutical compositions for the treatment of systemic sclerosis. In particular, the present invention is defined by the claims.

### DETAILED DESCRIPTION OF THE INVENTION

Systemic sclerosis (SSc) is an autoimmune disease characterized by fibrosis of the skin and visceral organs and vascular alterations. SSc pathophysiology involves systemic inflammation and oxidative stress. Since vanin-1 gene (vnn1) encodes an enzyme with pantetheinase activity that converts the vasculoprotective pantethine into profibrotic pantothenic acid and prooxidant cystamine, the inventors tested this pathway in the pathophysiology of SSc. The activation of the vanin-1/pantetheinase pathway was investigated in wild-type BALB/c mice with HOCl-induced-SSc by ELISA and western-blotting. The inventors then evaluated the effects of the invalidation of vnn1 on the development of fibrosis, endothelial alterations and immunological activation in mice with HOCl-induced SSc. They then explored the vanin-1/pantetheinase pathway in a cohort of patients with SSc and in controls. In wild-type mice with HOCl-induced-SSc, the vanin-1/pantetheinase pathway was dysregulated with elevation of skin vanin-1 activity and high levels of serum pantothenic acid. The invalidation of the vnn1 gene in HOCl-vnn1−/− mice prevented the development of characteristic features of the disease: fibrosis, immunologic abnormalities and endothelial dysfunction. Remarkably, patients with diffuse SSc also had an increased expression of vanin-1 in skin and blood, and elevated levels of serum pantothenic acid, that correlate with the severity of the disease. Our data demonstrate that vanin-1/pantetheinase controls fibrosis, vasculopathy, auto-immunity and oxidative stress in systemic sclerosis. The levels of vanin-1 expression and pantothenic acid determine SSc severity and could be used as markers of the severity of the disease. More importantly, the inhibition of vanin-1 could open new therapeutic approaches in SSc.

Accordingly a first object of the present invention relates to a method of treating systemic sclerosis in a subject in need thereof comprising administering to the subject a therapeutically effective amount of vanin-1 inhibitor.

As used herein, the term “subject” refers to a human or another mammal (e.g., mouse, rat, rabbit, hamster, dog, cat, cattle, swine, sheep, horse or primate). In some embodiments, the subject is a human being. The term “subject” does not denote a particular age, and thus encompass adults, children and newborns.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

In particular, the vanin-1 inhibitor of the present invention is particularly suitable for reducing fibrosis, vasculopathy (e.g. vascular stress or vascular dysfunction), inflammation, auto-immunity and oxidative stress in systemic sclerosis. In some embodiments, the vanin-1 inhibitor of the present invention is particular suitable for reducing fibrosis that affects at least one organ selected from the group consisting of skin, heart, liver, lung, or kidney. In some embodiments, the vanin-1 inhibitor of the present invention is particular suitable for reducing the level of anti-DNA topoisomerase-1 antibodies. In some embodiments, the vanin-1 inhibitor of the present invention is particular suitable for reducing endothelial activation. In some embodiments, the vanin-1 inhibitor of the present invention is particular suitable for reducing systemic oxidative stress. The method of the present invention is also particularly suitable for preventing pulmonary arterial hypertension.

As used herein, the term “vanin-1” has its general meaning in the art. Vanin-1 is a pantetheinase that catalyzes the hydrolysis of pantetheine to produce pantothenic acid (vitamin B5) and cysteamine. As used herein the term “vanin-1 inhibitor” refers to any compound that is able to inhibit the activity or expression of vanin-1. The term encompasses any vanin-1 inhibitor that is currently known, and/or any vanin-1 inhibitor that can be subsequently discovered or created, can be employed with the presently disclosed subject matter. More particularly the term “vanin-1 inhibitor” is preferably defined herein as a compound which, in vitro and/or in vivo: (i) inhibits the activity and/or expression of Vanin-1; and/or (ii) blocks processing of pantetheine into cysteamine and pantothenic acid; and/or (iii) blocks intracellular synthesis of cysteamine and/or of cystamine, the oxidized form of cysteamine. Inhibition and blocking may be total or partial. Methods for identifying whether a compound is a vanin-1 inhibitor are well known in the art (see e.g. Ruan B Hl, Cole D C, Wu P, Quazi A, Page K, Wright J F, Huang N, Stock J R, Nocka K, Aulabaugh A, Krykbaev R, Fitz L J, Wolfman N M, Fleming M L. A fluorescent assay suitable for inhibitor screening and vanin tissue quantification. Anal Biochem. 2010 Apr. 15; 399(2):284-92. doi: 10.1016/j.ab.2009.12.010. Epub 2009 Dec. 16.)

In some embodiments, the vanin-1 inhibitor of the present invention is small organic molecule. Several vanin-1 inhibitors have been identified and are well known in the art and typically include the compounds described in the International Patent Publication WO 2014048547 or in Jansen P. et al. Discovery of Small Molecule Vanin Inhibitors: New Tools To Study Metabolism and Disease ACS Chem. Biol. 2013, 8, 530-534.

In some embodiments, the vanin-1 inhibitor is:

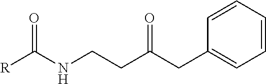

wherein

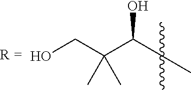 [SMILES: *[C@H](O)C(C)(C)CO]

In some embodiments, the vanin-1 inhibitor is selected from the group consisting of:

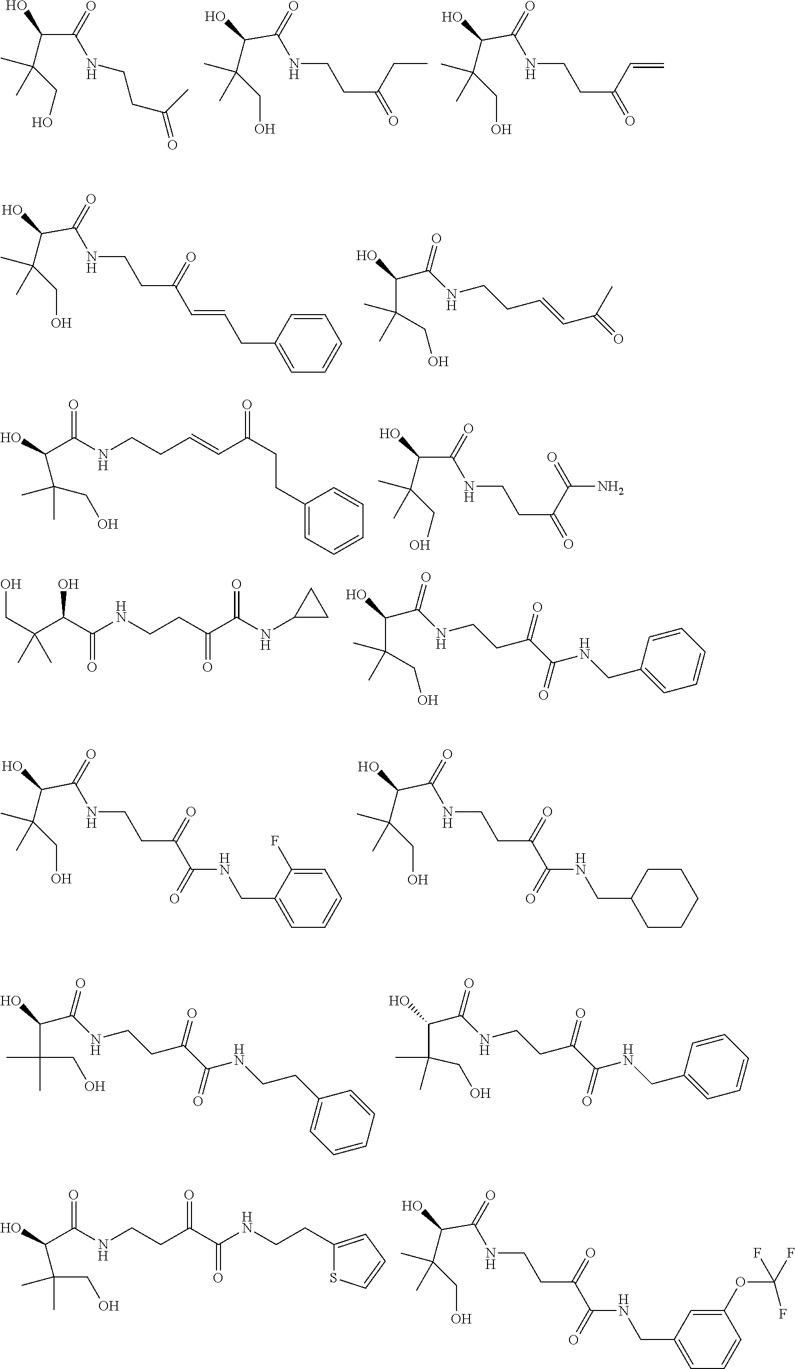

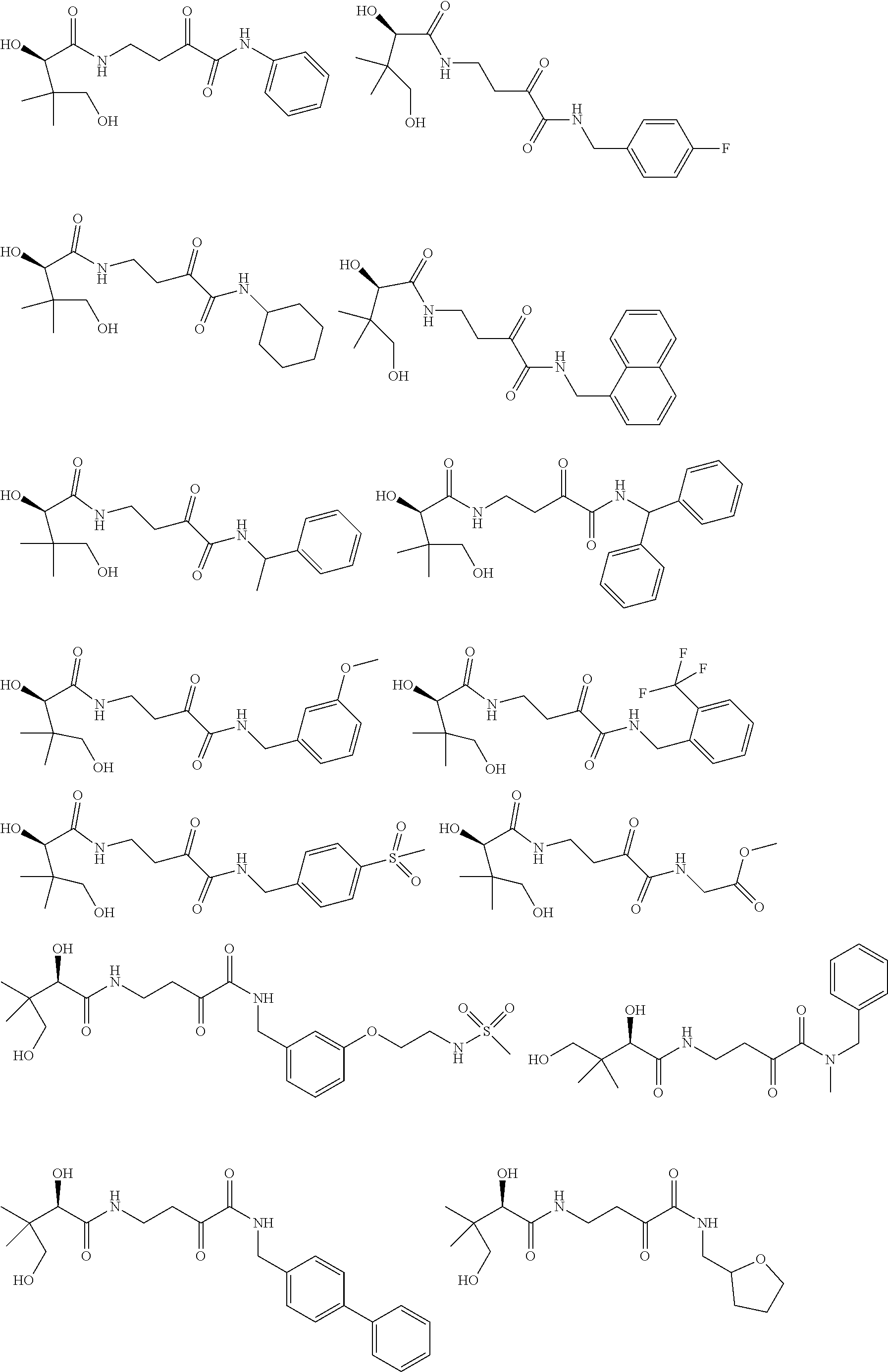

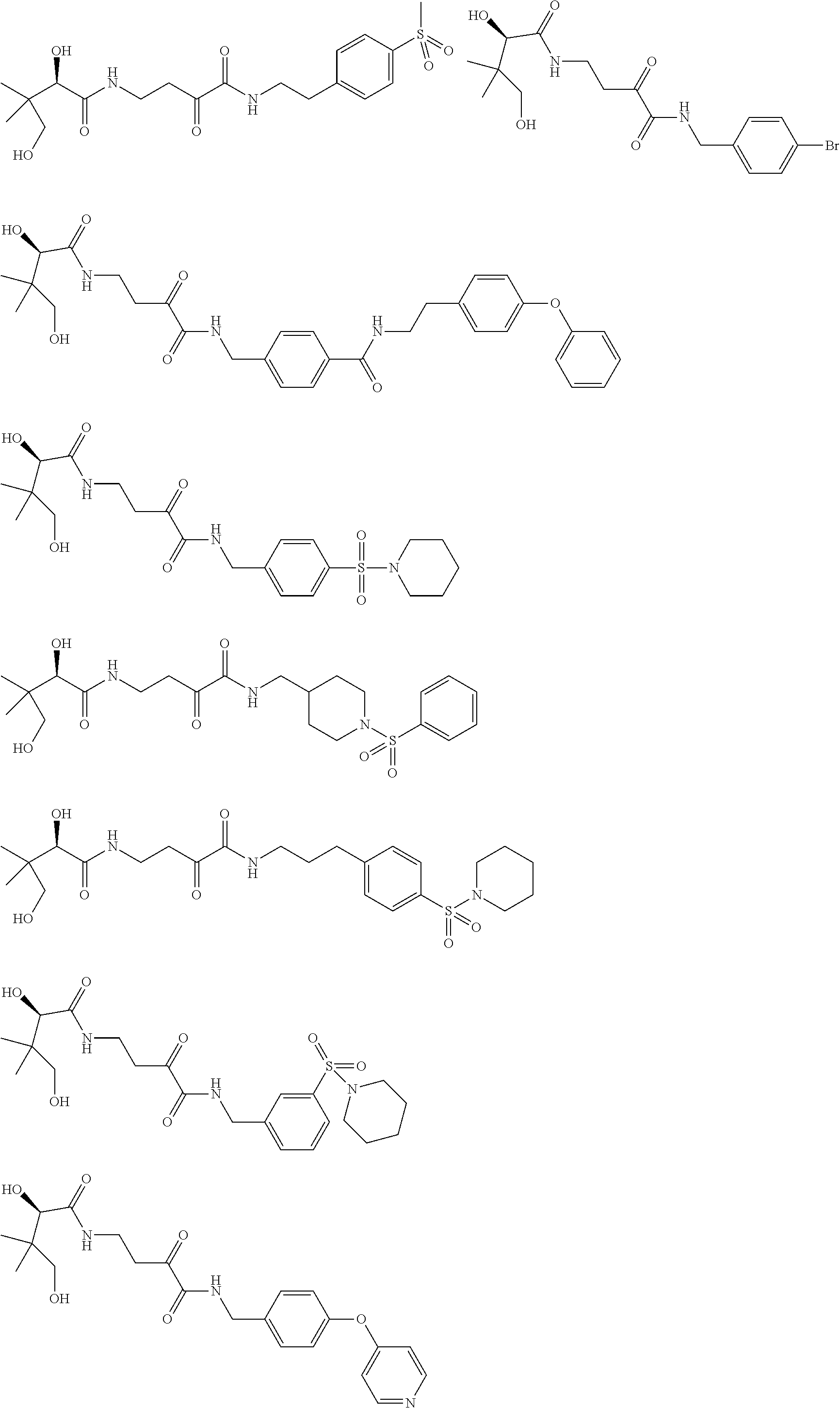

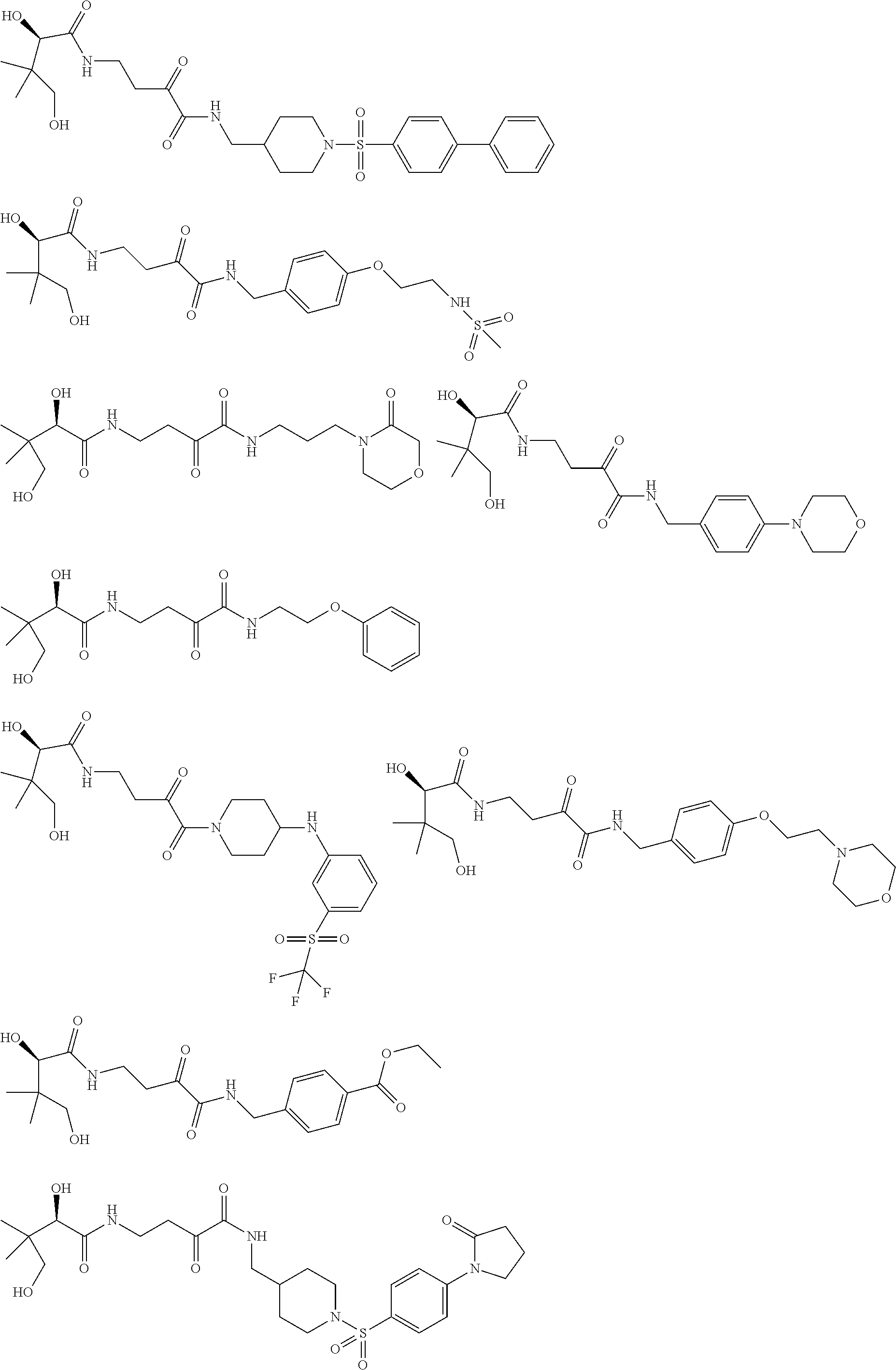

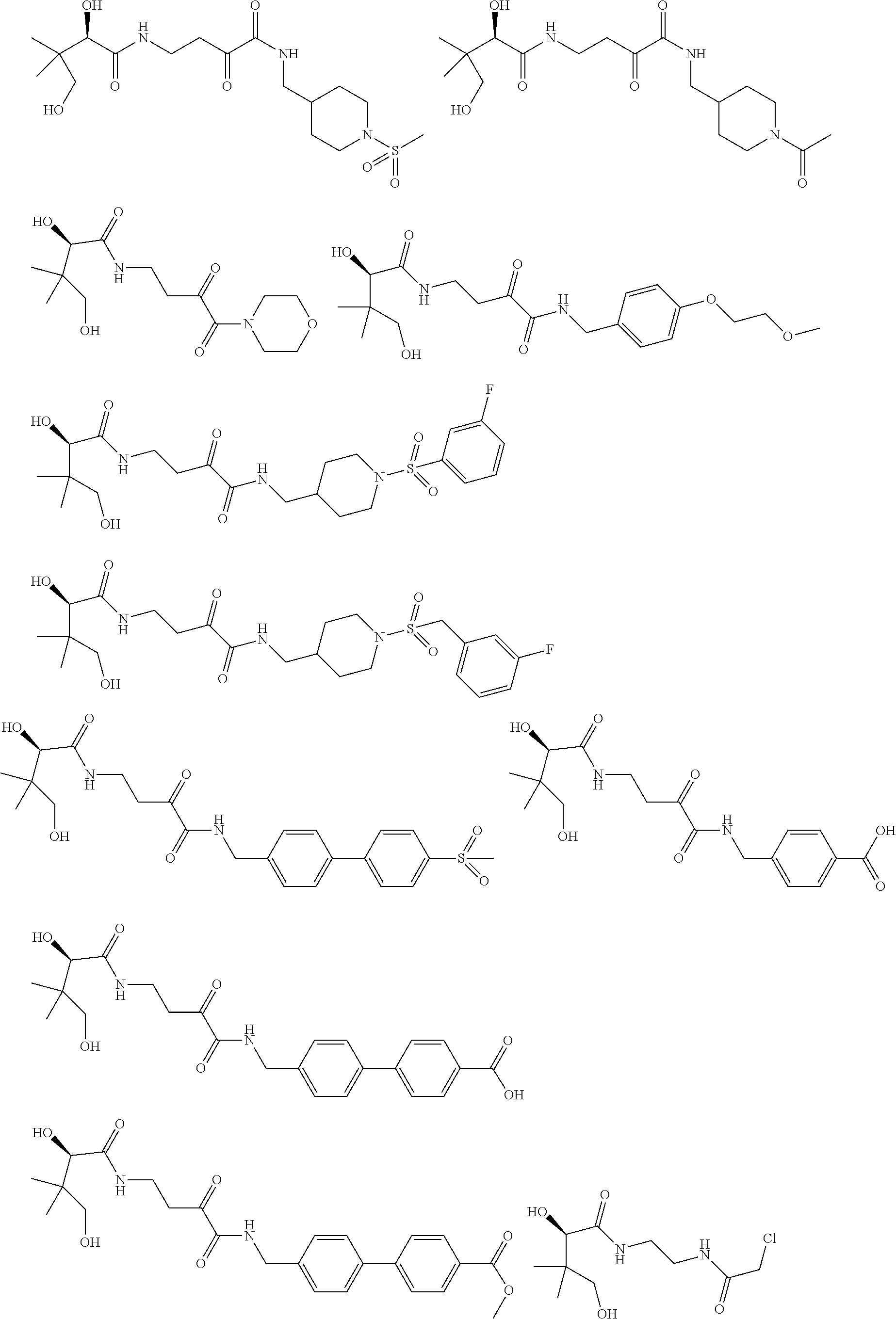

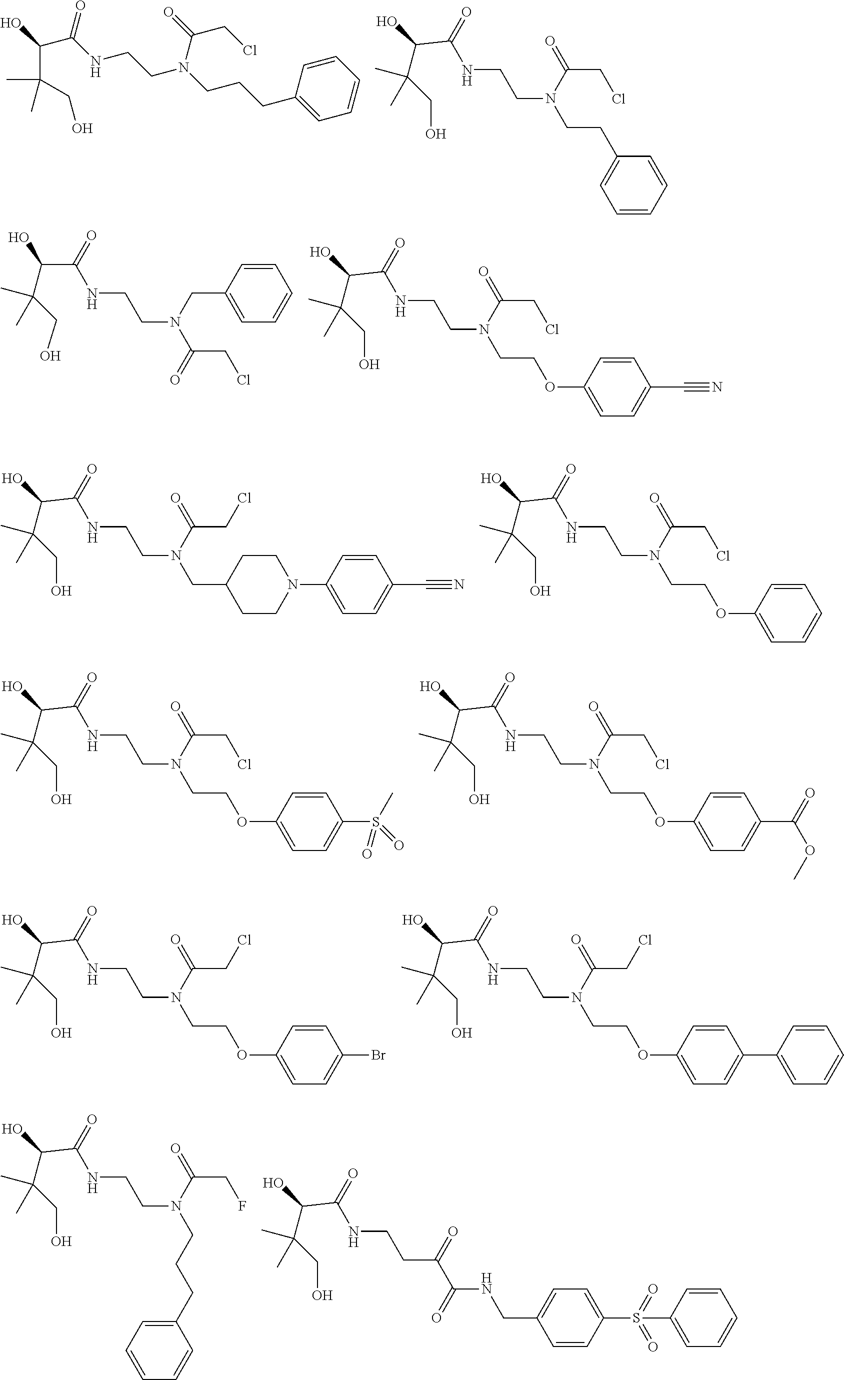

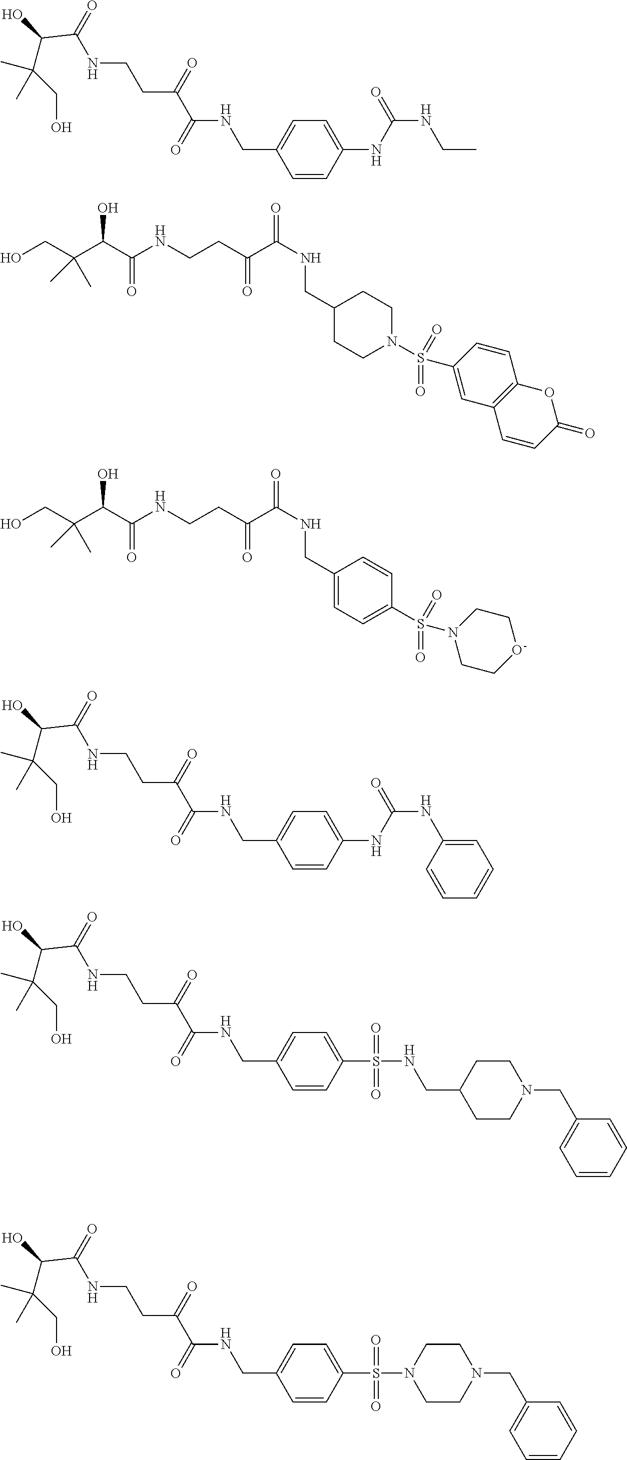

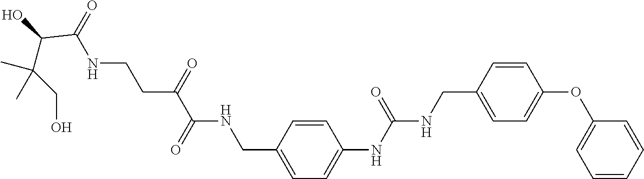 [SMILES: CC(C)(CO)[C@@H](O)C(=O)NCCC(=O)C(=O)NCc1ccc(NC(=O)NCc2ccc(Oc3ccccc3)cc2)cc1]

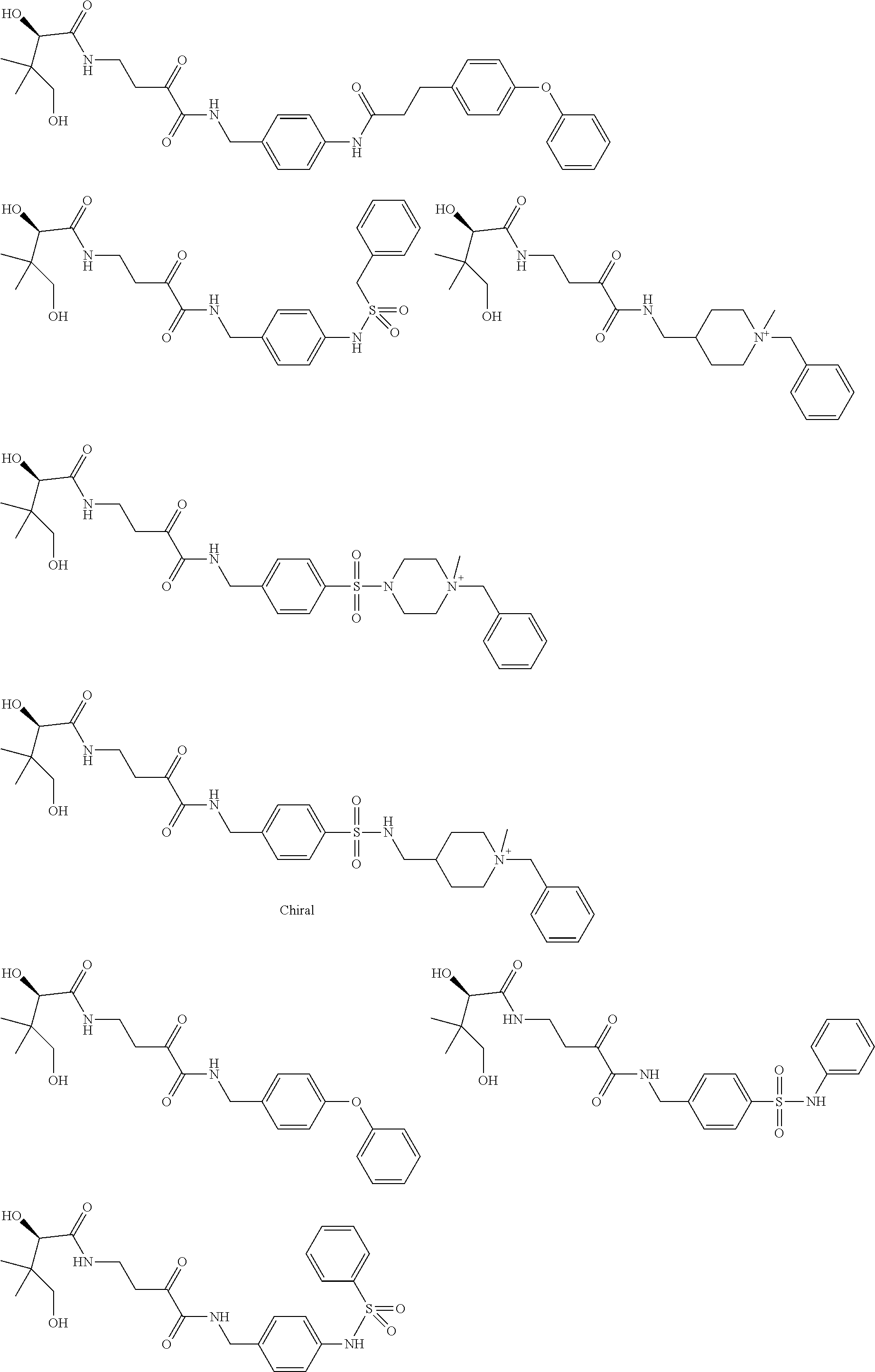

In some embodiments, the vanin-1 inhibitor is an antibody. As used herein, the term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies. In some embodiments, the antibody is a chimeric antibody a humanized antibody or a human antibody. In some embodiments, the antibody is a single domain antibody. As used herein, the term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In some embodiments, the vanin-1 inhibitor is an inhibitor of vanin-1 expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of vanin-1 mRNA by binding thereto and thus preventing protein translation or increasing mRNA degradation, thus decreasing the level of vanin-1, and thus activity, in a cell. For example, antisense oligonucleotides of at least about 15 bases and complementary to unique regions of the mRNA transcript sequence encoding vanin-1 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. vanin-1 gene expression can be reduced by contacting a subject or cell with a small double stranded RNA (dsRNA), or a vector or construct causing the production of a small double stranded RNA, such that vanin-1 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing vanin-1. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

By a “therapeutically effective amount” of the vanin-1 inhibitor as above described is meant a sufficient amount of the compound for treating systemic sclerosis. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the vanin-1 inhibitor is administered to the subject in the form of a pharmaceutical composition. Typically, the vanin-1 inhibitor may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The vanin-1 inhibitor can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

A further object of the present invention relates to a method of determining the severity of systemic sclerosis in a subject comprising i) determining the level of vanin-1 activity in a blood sample obtained from the subject ii) comparing the level determined at step i) with a predetermined reference value and iii) concluding that the subject suffers from a severe form of systemic sclerosis when the level determined at step i) is higher than the predetermined reference value or concluding that the subject suffers from limited form of systemic sclerosis.

Typically, subjects suffering from a severe form of systemic sclerosis include subject with a diffuse sclerosis, multiple or multiple sclerodermic lesion or even pulmonary arterial hypertension. Subjects suffering from a limited form of systemic sclerosis typically include subject with a cutaneous limited form.

As used herein, the term “blood sample” refers to a whole blood sample, serum sample and plasma sample. A blood sample may be obtained by methods known in the art including venipuncture or a finger stick. Serum and plasma samples may be obtained by centrifugation methods known in the art. The sample may be diluted with a suitable buffer before conducting the assay.

The measurement of the level of vanin-1 activity in the blood sample is typically carried out using standard protocols known in the art. Typically, said measurement includes the determination of the vanin-1 level or pantothenic acid in the blood sample. For example, the method may comprise contacting the blood sample with a binding partner capable of selectively interacting with vanin-1 in the sample. In some embodiments, the binding partners are antibodies, such as, for example, monoclonal antibodies or even aptamers. For example the binding may be detected through use of a competitive immunoassay, a non-competitive assay system using techniques such as western blots, a radioimmunoassay, an ELISA (enzyme linked immunosorbent assay), a “sandwich” immunoassay, an immunoprecipitation assay, a precipitin reaction, a gel diffusion precipitin reaction, an immunodiffusion assay, an agglutination assay, a complement fixation assay, an immunoradiometric assay, a fluorescent immunoassay, a protein A immunoassay, an immunoprecipitation assay, an immunohistochemical assay, a competition or sandwich ELISA, a radioimmunoassay, a Western blot assay, an immunohistological assay, an immunocytochemical assay, a dot blot assay, a fluorescence polarization assay, a scintillation proximity assay, a homogeneous time resolved fluorescence assay, a IAsys analysis, and a BIAcore analysis. The aforementioned assays generally involve the binding of the partner (ie. antibody or aptamer) to a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like. An exemplary biochemical test for identifying specific proteins employs a standardized test format, such as ELISA test, although the information provided herein may apply to the development of other biochemical or diagnostic tests and is not limited to the development of an ELISA test (see, e.g., Molecular Immunology: A Textbook, edited by Atassi et al. Marcel Dekker Inc., New York and Basel 1984, for a description of ELISA tests). Therefore ELISA method can be used, wherein the wells of a microtiter plate are coated with a set of antibodies which recognize vanin-1. A sample containing or suspected of containing vanin-1 is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art. Measuring the level of vanin-1 (with or without immunoassay-based methods) may also include separation of the compounds: centrifugation based on the compound's molecular weight; electrophoresis based on mass and charge; HPLC based on hydrophobicity; size exclusion chromatography based on size; and solid-phase affinity based on the compound's affinity for the particular solid-phase that is used. Once separated, said one or two biomarkers proteins may be identified based on the known “separation profile” e.g., retention time, for that compound and measured using standard techniques. Alternatively, the separated compounds may be detected and measured by, for example, a mass spectrometer. Typically, levels of immunoreactive vanin-1 in a sample may be measured by an immunometric assay on the basis of a double-antibody “sandwich” technique, with a monoclonal antibody specific for vanin-1 (Cayman Chemical Company, Ann Arbor, Mich.). According to said embodiment, said means for measuring vanin-1 level are for example i) a vanin-1 buffer, ii) a monoclonal antibody that interacts specifically with vanin-1, iii) an enzyme-conjugated antibody specific for vanin-1 and a predetermined reference value of vanin-1.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of level of vanin-1 in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the levels of vanin-1 in a group of reference, one can use algorithmic analysis for the statistic treatment of the measured levels of vanin-1 in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

The predetermined reference value can also be relative to a number or value derived from population studies, including without limitation, subjects of the same or similar age range, subjects in the same or similar ethnic group, and subjects having the same severity of systemic sclerosis. Such predetermined reference values can be derived from statistical analyses and/or risk prediction data of populations obtained from mathematical algorithms and computed indices. In some embodiments, the predetermined reference values are derived from the level of vanin-1 in a control sample derived from subjects having different severity of systemic sclerosis. Furthermore, retrospective measurement of the level of vanin-1 in properly banked historical subject samples may be used in establishing these predetermined reference values.

In some embodiments, a cut-off value thus consists of a range of quantification values, e.g. centered on the quantification value for which the highest statistical significance value is found. For example, on a hypothetical scale of 1 to 10, if the ideal cut-off value (the value with the highest statistical significance) is 5, a suitable (exemplary) range may be from 4-6. For example, a subject may be assessed by comparing values obtained by measuring the level of vanin-1, where values greater than 5 reveal that the subject suffers from a severe form of systemic sclerosis and values less than 5 reveal that the subject suffers from a limited form of systemic sclerosis. In some embodiments, a subject may be assessed by comparing values obtained by measuring the level of vanin-1 and comparing the values on a scale, where values above the range of 4-6 indicate that the subject from a severe form of systemic sclerosis and values below the range of 4-6 indicate that the subject suffers from a limited from of systemic sclerosis, with values falling within the range of 4-6 indicate that the subject suffers from a mild form of systemic sclerosis.

In some embodiments, determining the level of vanin-1 activity in the blood sample is suitable for determining whether the subject shall be eligible to a treatment with a vanin-1 inhibitor as above described. Accordingly a further object of the present invention a method of treating systemic sclerosis in a subject in need thereof comprising i) determining the level of vanin-1 activity in a blood sample obtained from the subject ii) comparing the level determined at step i) with a predetermined reference value and iii) administering to the subject a therapeutically effective amount of a vanin-1 inhibitor when the level determined at step i) is higher than the predetermined reference value.

The invention will be further illustrated by the following figures and examples. However, these examples and figures should not be interpreted in any way as limiting the scope of the present invention.

### FIGURES

FIG. 1. Vanin-1 is implicated in the development of SSc in mice and vnn1^{-/-}-mice are more resistant to HOCl-induced inflammation than WT-mice. (A) Levels of vanin-1 activity in skin fibroblasts from PBS and HOCl-BALB/c mice measured with the AMC substrate. (B) Levels of expression of Vanin-1 in skin fibroblasts from PBS and HOCl-BALB/c mice by western-blot (3 mice per group representative of 7). (C) Serum pantothenic acid concentrations in PBS- and HOCl-BALB/c mice (n=12 per group). Mean values were compared using paired Mann Whitney U-tests. *P<0.05; **P<0.01***P<0.001.NS: non significant differences.

FIG. 2. Vnn1^{-/-}-mice are more resistant to HOCl-induced fibrosis and inflammation than WT-mice. WT and vnn1^{-/-}-mice were submitted to the protocol of HOCl-induced SSc (n=7 per group). (A) Dermal thickness in injected skin areas at week 6 (mm). (B, C) Hydroxyproline content in 6 mm punch skin biopsies and in lung (mg/punch and mg/lobe). (D) Serum pantothenic acid concentrations in the sera (μG/L). (E) Alpha-SMA expression levels measured by western-blot (2 mice representative of 7 per group). Black lines indicate a grouping of images from different parts of the same gel. (Vnn1) Vanin-1. Mean values were compared using paired Mann Whitney U-tests. *P<0.05; **P<0.01***P<0.001.NS: non significant differences.

FIG. 3 (A) Serum anti-DNA-topoisomerase-1 Ab levels as measured by ELISA (AU). (B, C, D, E) Levels of IL-4, IL-13, IL-17 and IFN-γ in CD4 T-lymphocytes supernatants (pg/mL). Vnn1: Vanin-1. Mean values were compared using paired Mann Whitney U-tests. *P<0.05; **P<0.01***P<0.001.NS: non significant differences.

FIG. 4. (A) Serum sVCAM levels measured by ELISA (ng/mL). (B) Serum AOPP levels (AU per μmol of chloramine-T equivalents). (C) Total thiols levels in the sera (μM). (Vnn1) Vanin-1. Mean values were compared using paired Mann Whitney U-tests. *P<0.05; **P<0.01***P<0.001.NS: non significant differences.

FIG. 5. The pantethine/vanin-1 pathway is dysregulated in SSc patients. (A-C) vanin-1 levels measured by ELISA in sera from healthy subjects and SSc patients (μG/mL). (D) Immunostaining of vanin-1 in skin biopsies from 2 healthy subjects (1 and 2) and 2 SSc patients (3 and 4) representative of 4 patients. (E-G) Serum pantothenic acid concentration in healthy subjects and SSc patients (μg/L). (dSSc and lSSc) Diffuse and limited systemic sclerosis, (PAHT) pulmonary arterial hypertension. Mean values were compared using paired Mann Whitney U-tests. *P<0.05, **P<0.01***P<0.001. NS: non significant differences.

FIG. 6. Schematic description of the role of the vanin-1 pathway in the development of inflammation, fibrosis and vascular damages in SSc.

### EXAMPLE

Material & Methods:

Mice.

Six week-old female BALB/c mice were purchased from Harlan (Gannat, France). Vnn1^{-/-} BALB/c mice were generated in the laboratory of P. Naquet as previously described (21). In all experiments, control mice were age-, sex- and weight-matched with experimental mice. All animals were maintained with food and water ad libitum and were given humane care according to the guidelines of our institution. The project has received the approval of the Regional Ethics Committee on Animal Experimentation under the number CEEA34.CN.023.11.

Determination of Vanin-1/Pantetheinase Levels and Activity.

The Vanin-1 ELISA and the serum pantetheinase activity assays were previously described (25). For the enzyme assay, 20 μl pre-reduced serum (50 μM 2-beta-mercaptoethanol, 10 minutes) were added to 20 μl of pPNA (0.2 mg/ml) in sodium phosphate buffer 10 mM, pH 8.0, and the increase in absorbance (405 nm) measured (c=12144 M⁻¹ cm¹).

Measurement of Serum Pantothenic Acid Levels.

The levels of serum pantothenic acid were determined using the Pantothenic Acid kit according to the manufacturer's instructions (Servibio, France).

In Vivo Induction of Experimental SSc and Treatments.

HOCl-induced SSc developed following daily intradermal injections of 2×200 μl of HOCl-generating reagents into the back of BALB/c female mice for 6-weeks, as previously described (n=28 mice) (6). Control groups received injections of 200 μl sterilized PBS (n=28 mice). Three days after the end of injections and treatment, the animals were sacrificed by cervical dislocation. Lungs were collected and skin biopsies were performed on the back region with a punch (6 mm diameter). Samples were stored at −80° C. Induction of SSc in Vnn1^{-/-} and wild-type female mice was performed according to the same protocol.

Assessment of Skin Thickness and Collagen Content by Hydroxyproline Assay Content.

Skin thickness of the shaved back of mice was measured with a caliper and expressed in millimeters one day before the sacrifice of the animal. Fixed lung and skin pieces were embedded in paraffin. A 5-μm-thick tissue section was prepared from the mid-portion of paraffin-embedded tissue and stained with picrosirius red. Slides were examined by standard brightfield microscopy (Nikon Eclipse 80i, France) by a pathologist who was blinded to the assignment of the animal to an experimental or a control group. The collagen content in lesional skin and lung samples was explored by hydroxyproline assay (29). Skin taken from the site of injection (6-mm punch biopsies) and lung pieces (same lobe for each mouse) were diced using a sharp scalpel, put into aseptic tubes, thawed and digested in 6 M HCl for three hours at 120° C. Samples were then mixed with 0.06 M chloramine T and incubated for 20 min at room temperature. Next, 3.15 M perchloric acid and 20% p-dimethylaminobenzaldehyde were added and samples were incubated for additional 20 min at 60° C. The absorbance was determined at 557 nm with a microplate reader (Fusion, PerkinElmer, Wellesley, USA).

Primary Fibroblasts.

Normal and SSc primary skin fibroblasts were isolated from mouse skin as previously described (30). Skin fragments from the back of mice were collected at the time of sacrifice. Skin samples were digested with “Liver Digest Medium” (Invitrogen) for 1 hour at 37° C. After three washes in complete medium, cells were seeded into sterile flasks and isolated fibroblasts were cultured in DMEM/Glutamax-I supplemented with 10% heat-inactivated fetal calf serum and antibiotics at 37° C. in humidified atmosphere with 5% CO₂. Only primary fibroblasts were used in this study.

Western-Blot Analysis of α-SMA and Vanin-1 Proteins in Mouse Fibroblasts.

Fibroblasts isolated as above were incubated with 50 μl RIPA. Protein extracts (30 μg total proteins) were subjected to a 10% polyacrylamide gel electrophoresis, transferred onto nitrocellulose membranes, blocked with 5% non-fat dry milk in Tris Buffer Solution-Tween, then incubated overnight at 4° C. with an anti-α-SMA antibody ((Sigma Aldrich, St Quentin Fallavier, France) or an anti-vnn-1 antibody (mAb34G7, kindly given by Pr P. Naquet). The membranes were washed in TBST and incubated with a HRP-conjugated secondary antibody (Santa-Cruz, Paris, France) for one hour at room temperature. Immunoreactivities were revealed with ECL (Amersham) and membranes were developed with a Fujifilm developer (Fujifilm, France).

Assays of sVCAM in Sera.

Levels of sVCAM in sera were measured by ELISA using the Mouse sVCAM/CD106 Duoset kit (R&D Systems, Lille, France) following the manufacturer's instructions.

Measurement of Advanced Oxidized Protein Products (AOPP) in Sera.

AOPP were measured by spectrophotometry as previously described (5). The assay was calibrated using Chloramine-T. The absorbance was determined at 340 nm on a microplate reader (Fusion, PerkinElmer, Wellesley, USA). AOPP concentrations were expressed as μmol/L of chloramine-T equivalents.

Measurement of Total Thiols in Sera.

Thiol determinations were based on the thiol/disulfide reaction of thiol and Ellman's reagent, 5,50-dithiobis(2-nitrobenzoic acid) DTNB (Sigma Aldrich, St. Louis, Mo., USA). Fifty μL of the sample mixed with 1 mL 0.1 M Tris, 10 mM EDTA pH 8.2, constituting the blank reaction, was assessed at 412 nm (UVIKON). The addition of 40 μL 10 mM DTNB in methanol triggered the reaction and absorption at 412 nm was measured after stable colour formation (1-3 min). The concentrations of thiol groups were calculated using a molar extinction coefficient of 13,600/M/cm. Thiols were expressed as μmol/L.

Assays for Serum Anti-DNA Topoisomerase 1 Autoantibodies.

Levels of anti-DNA topoisomerase 1 IgG Abs were detected by enzyme-linked immunosorbent assay (ELISA) using purified calf thymus DNA topoisomerase I bound to the wells of a microtiter plate (Abnova, Germany). A 1:4 mice serum dilution and a 1:1000 anti-murine Ig HRP (DAKO) secondary antibody dilution were used.

T Lymphocyte Production of IL-4, IL-13, IL-17 and IFN-γ by ELISA.

Spleen cells were cultured in RPMI-1640 supplemented with antibiotics, Glutamax (Invitrogen Life Technologies), and 10% heat-inactivated FCS (Invitrogen Life Technologies) (complete medium). CD4 T cells were isolated from spleen cell suspensions by a positive selection using CD4 microbeads and LS columns (Miltenyi Biotec, France) according the manufacturer's instructions. CD4 T cell suspensions were then seeded in 24-well flat-bottom plates and cultured (2×10⁶ cells) in complete medium for 72 h in the presence of 5 μg/mL concanavalin A. Supernatants were collected and cytokine concentrations (IL-4, IL-13, IL-17 and IFN-γ) were determined by ELISA according to the manufacturer's instructions (Ebioscience, San Diego, USA). Results were expressed in pg/mL.

Patients.

Fifty-one patients and thirty healthy subjects were enrolled in the study (Table 1). They signed informed consent forms and sera (for all patients) or skin biospsies (only for 4 patients) were collected. To be eligible for the study, patients had to fulfill the American College of Rheumatology criteria and/or the Leroy & Medsger criteria for SSc (31, 32).

Immunohistochemical Vanin-1 Staining.

The immunostaining was performed as previously described with minor modifications (33). Skin biopsies from experimental and control patients were fixed, embedded in paraffin, sectioned and stained for light microscopy. Immunohistochemical studies were performed using the indirect immuno-peroxidase staining technique on a paraffin-embedded section with haematoxylin counterstain. Paraffin sections (4 μm) were cut on the day of immunostaining. Sections were placed onto poly-L-lysine-coated slides (Superfrost II®) and deparaffinized. After blocking of endogenous peroxidase, sections were incubated overnight at 4° C. with a rat anti-Vanin-1 mAb34G7 (33) diluted 1:100 in antibody diluent (DAKO), followed by 30 minutes' incubation with a rabbit anti-rat immunoglobulin HRP conjugated IgG Ab. The complex was then visualized with a hydrogen peroxide substrate and 3,3′-diaminobenzidine tetrahydrochloride as the chromogen. The slides were rinsed with a Tris-based buffer solution, counter-stained with haematoxylin. Negative controls were prepared by replacing the primary antibody with normal rat serum. Slides were analyzed by two pathologists.

Statistical Analysis.

All quantitative data were expressed as means±SEM. Data were compared using the Mann-Whitney nonparametric test. When analysis included more than 2 groups, one way ANOVA analysis of variance with the Bonferroni post-test was used. P value <0.05 was considered significant. Statistical analysis was performed with Graphpad Prism 4.0 (La Jolla, USA)

Results:

Vanin-1 Activity in Skin Fibroblasts and Serum Pantothenic Acid are Higher in Mice with HOCl-Induced SSc than in Controls.

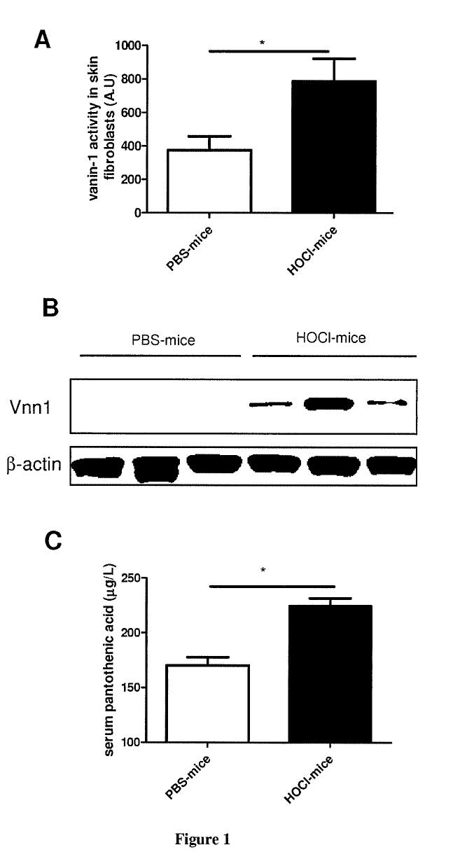

Vanin-1 is a membrane-bound ubiquitous enzyme with pantetheinase activity that converts the reduced form of pantethine, pantetheine, into pantothenic acid and cysteamine. The levels of vanin-1 activity measured with the AMC substrate in skin fibroblasts, and its expression assessed by western-blotting on fibroblast extracts, were higher in HOCl-mice than in control mice (P=0.007, FIG. 1A-B). The dysregulation of the vanin-1/pantethine pathway in HOCl-mice was also supported by the increase in serum concentration of pantothenic acid in HOCl-mice versus control-mice (P<0.0001, FIG. 1C).

The Clinical and Histological Features of Fibrosis are not Observed in HOCl-Vnn-1^{-/-} Mice.

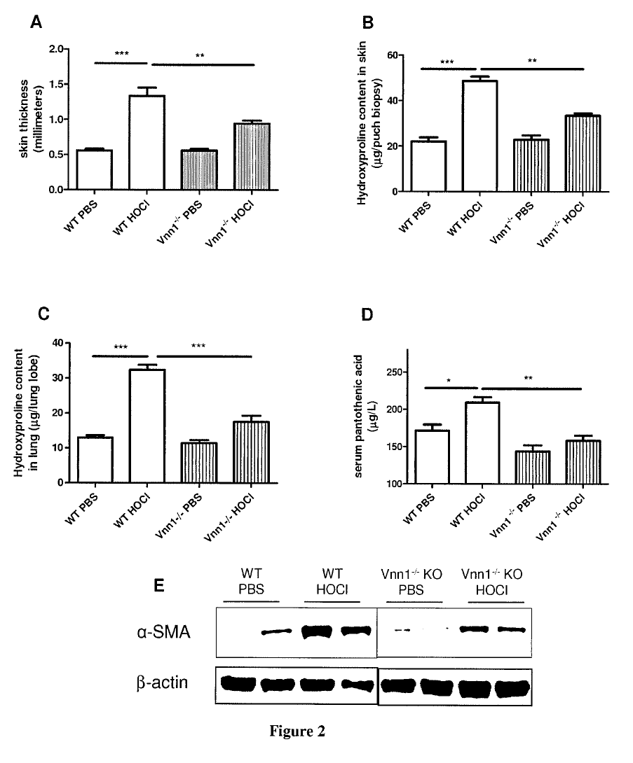

SSc was induced in Vanin-1 null mice (vnn1^{-/-}) using the same protocol as in the wild-type (HOCl-WT-mice). These mice showed a milder skin thickening than wild-type mice (P=0.0137, FIG. 2A). HOCl-induced skin and lung fibrosis were significantly reduced in vnn-1^{-/-} mice compared to wild-type mice as shown by the hydroxyproline content in both tissues (P=0.0017 in skin and P=0.0006 in lungs, FIG. 2B, C). Alpha-SMA, a marker of myofibroblast differentiation, was expressed at a lower level in fibroblasts from HOCl-vnn1^{-/-} mice than in those from HOCl-WT-mice (FIG. 2E). Furthermore, HOCl-vnn1^{-/-}-mice had significantly higher levels of profibrotic pantothenic acid than HOCl-WT-mice (P=0.019 between HOCl-WT-mice and HOCl-vnn1^{-/-}-mice, FIG. 2D).

Invalidation of the Vnn1 Gene Reduced the Immunological Activation in HOCl-Mice.

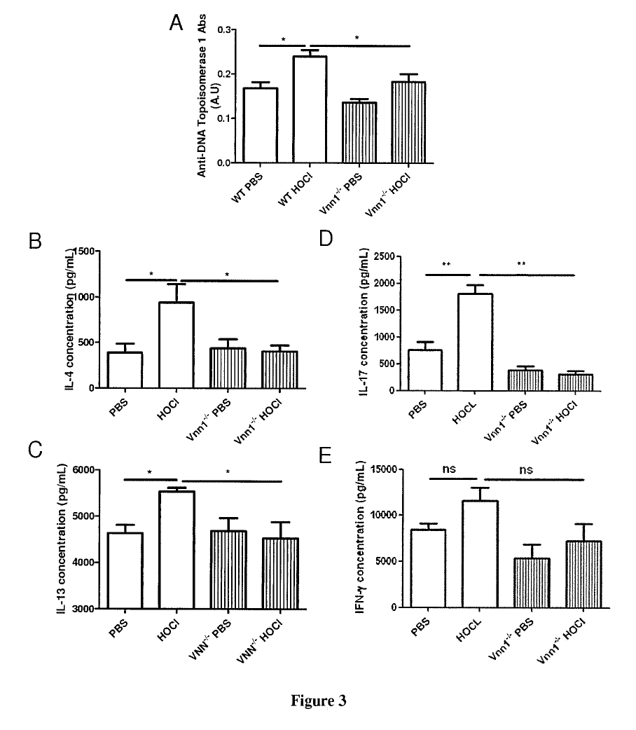

The levels of serum anti-DNA topoisomerase-1 antibodies, a hallmark of human SSc and mouse HOCl-induced SSc, were lower in HOCl-vnn1^{-/-}-mice than in HOCl-WT-mice (P=0.047 for anti-DNA topoisomerase-1 Abs, FIG. 3A). We explored the splenic CD4 T cell production of IL-4, IL-13, IL-17 and IFN-γ in WT- and vnn1^{-/-}-mice injected with HOCl. Remarkably, T cells from vnn1^{-/-}-mice produced less IL-4, IL-13 and IL-17 following HOCl injections than WT mice (P=0.026, P=0.038 and P=0.0079 respectively, FIG. 3B, C). Splenic T cells from HOCl-vnn1^{-/-}-mice seems to produce less IFN-γ than HOCl-WT-mice, but this result did not reach significance (P=0.111)

Endothelial Activation and Systemic Oxidative Stress were not Present in vnn1^{-/-} Mice Injected with HOCl.

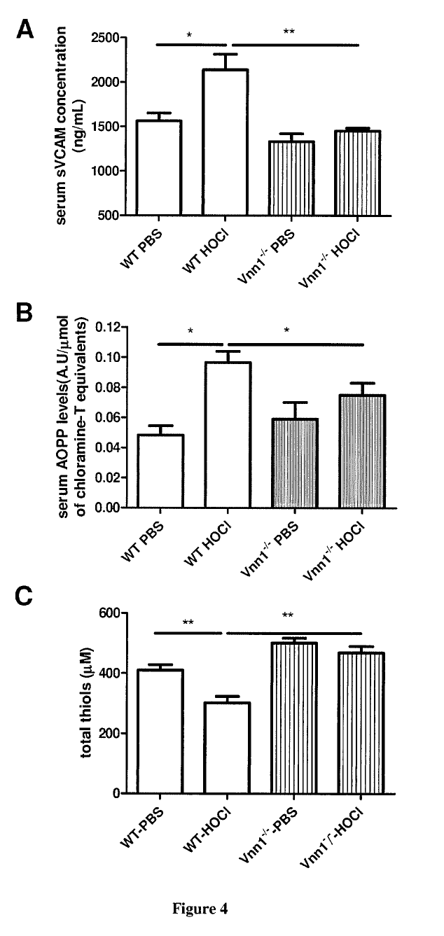

The endothelial activation in SSc is reflected by the release of soluble VCAM (Vascular Cell Adhesion Molecule) by endothelial cells. In contrast to HOCl-WT-mice, the levels of sVCAM remained at their initial level in HOCl-vnn1^{-/-}-mice (P=0.0012, FIG. 4 A). Serum AOPP, a marker of systemic oxidative stress, are elevated in WT-mice with HOCl-induced SSc compared to control PBS-WT-mice (P=0.0006, FIG. 4B). Serum AOPP in HOCl-Vnn-1^{-/-}-mice were significantly reduced by more than 20% compared to HOCl-WT-mice (P=0.049, FIG. 4B). In relation with seric AOPP levels, the concentration of total thiols in the serum is decreased by 25% in HOCl-WT-mice compared to PBS-WT-mice (P=0.037, FIG. 4C). We found that the invalidation of the vnn-1 gene allowed an elevation of total thiols level by 35% in HOCl-vnn-1^{-/-}-mice compared to HOCl-WT-mice (P=0.031, FIG. 4C).

Vanin-1 Inactivation Also Limits the Development of SSc in the Bleomycin Model

The inventors also demonstrated that the same beneficial effects on the development of SSc were observed in vnn1−/− mice treated with bleomycin compared with WT mice treated with bleomycin (40). WT mice receiving bleomycin developed fibrosis of the skin, as shown by levels of hydroxyproline concentration and collagen-1 and a-SMA mRNAs in skin biopsies (40). Interestingly, inactivation of the vnn1 gene had a beneficial effect on bleomycin induced fibrosis, because the hydroxyproline content, collagen-1 mRNA, and a-SMA mRNA levels were reduced significantly in vnn1−/− mice (40). This effect also was observed in lungs, because the hydroxyproline content and collagen-1 and a-SMA expression were decreased in vnn1−/− mice receiving bleomycin compared with WT mice receiving bleomycin (40).

Moreover, to assess the effect of vnn1 deletion on the systemic oxidant and inflammatory component of the disease, we measured the total thiol and IL-6 levels in the sera in this model. WT-mice with bleomycin-induced SSc had a slight reduction in total thiol levels and elevated levels of IL-6 compared with WT PBS mice (40). Deletion of the vnn1 gene had a beneficial effect on the oxidative response and inflammation in mice treated with bleomycin because the levels of total thiols and IL-6 in vnn1−/− mice with bleomycin-induced SSc reached those of PBS mice.

Skin and Serum Vanin-1 Activity and Serum Pantothenic Acid were Elevated in Patients with SSc

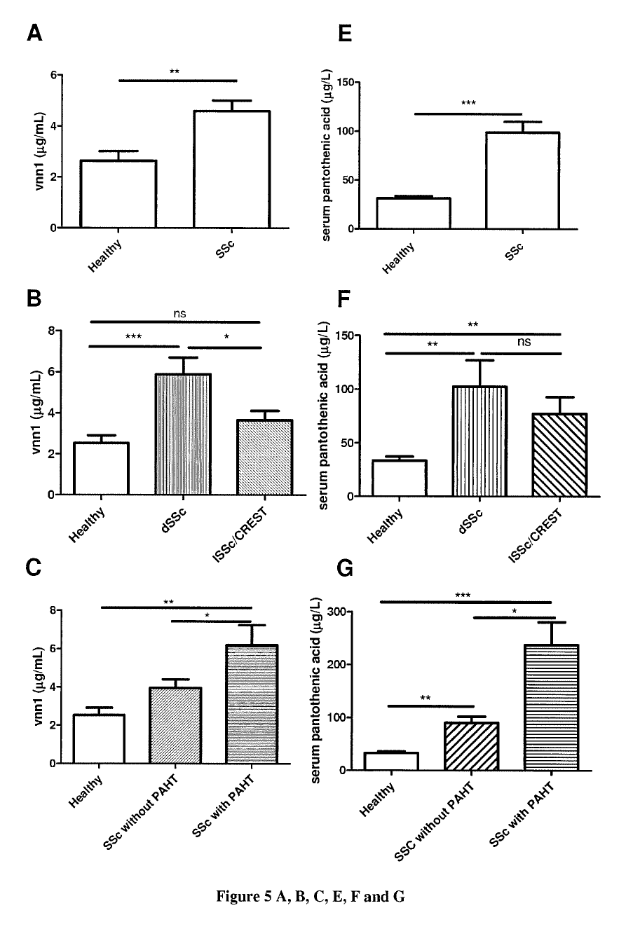

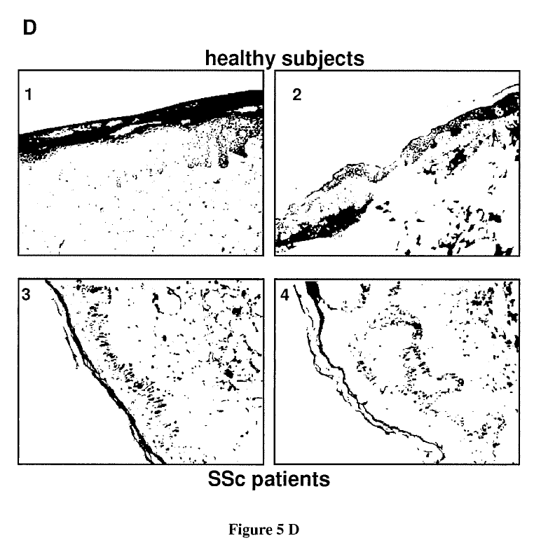

The mean level of vanin-1 in the sera from 51 patients with SSc was higher than in 30 healthy subjects (4,65±0.38 μg/mL versus 2.53±0.35 μg/mL, P<0.0001, FIG. 5A). Interestingly, the mean level of vanin-1 in sera from patients with diffuse SSc exceeded by 40% that of patients with a cutaneous limited form (5.873±0.82 μg/mL versus 3.654±0.45 μg/mL, P=0.0114, FIG. 5B). Similarly, in patients with SSc and pulmonary arterial hypertension, the mean serum level of vanin-1 activity was higher than in patients with normal pulmonary arterial pressure (6.33±1.002 μg/mL versus 4.133±0.39 μg/mL, p=0.0427, FIG. 5C).

Immunohistological staining of the samples of human SSc skin tested with anti-vanin-1 antibody, revealed a strong cytoplasmic staining in the basal epidermal layer and stratum germinativum (>1 cell/3) and a diffuse staining of the dermis. Vanin-1 staining was negative in control skins with no sclerodermic lesions (FIG. 5D).

Furthermore, serum pantothenic acid was higher in the 51 SSc-patients than in the 30 healthy subjects tested (97.8±11.2 μg/L versus 31.3±3.6n/L, P<0.0001, FIG. 5E). The serum levels of pantothenic acid were higher in patients with diffuse SSc than in patients with a limited/CREST form of SSc, (134.2±17.59 versus 78.11±14.07, FIG. 5F). SSc patients with pulmonary hypertension had the highest serum concentrations of pantothenic acid of all SSc-patients (mean of 237.5±433.23 μg/L in SSc-patients with PAHT, versus 90.1±12.01 μg/L in SSc-patients with no PAHT, P=0.018, FIG. 5G).

### DISCUSSION

We report that the hyperactivation of the vanin-l/pantetheinase pathway in SSc plays a role in the development of fibrosis, vasculopathy and auto-immune alterations that characterize the disease.

Given the relationship between inflammatory and oxidative stresses and hyperactivation of vanin-1 (20, 22), we first investigated the activity of vanin-1 in a murine model of SSc induced by oxidative stress. We observed a hyperactivation of vanin-1 in the skin of these diseased mice, which is a direct consequence of the chronic inflammatory process generated by HOCl-injections. Furthermore, this result is in line with the elevation of serum pantothenic acid that results from the hydrolysis of reduced pantethine (34). Since pantothenic acid is a profibrotic agent that elicits fibroblast activation and collagen synthesis (19), its involvement in the development of SSc is consistent with what is known of the fibrogenesis in this model.

In addition, the activation of the vanin-1/pantetheinase pathway also triggers oxidative stress through the generation of cystamine that inhibits gamma-glutamyl-cysteine-synthase and depletes GSH stores, thus allowing free radical overproduction beyond the antioxidant defence capacities (16). This is consistent with previous observations showing that fibroblasts of human and murine SSc display a reduced GSH store and an elevated production of ROS (6). The measurement of the elevation of cystamine levels in HOCl-mice would have been informative in our study, but the assay could not be performed in mice for technical reasons (35). However, as cystamine reduces thiols levels, the drop in thiol concentration observed in HOCl-mice is consistent with an elevated level of cystamine linked to the hyperactivation of vanin-1 in those mice.

Since the vanin-1/pantetheinase pathway is at the crossroads of inflammation, oxidative stress, fibrosis and endothelial alterations, we were then prompted to investigate the causal relationship between hyperactivation of the vanin-1/pantetheinase pathway and the development of SSc in the HOCl-model.

For that purpose, vnn1^{-/-}-mice were subjected to the induction of SSc by daily injections of HOCl. HOCl-vnn1^{-/-} mice displayed a markedly milder form of the HOCl-induced SSc when compared to HOCl-WT mice, with reduced fibrosis, and less pronounced markers of systemic oxidative and vascular stresses, and auto-immunity.

Indeed, the invalidation of vnn1 favors the accumulation of its substrate pantethine/pantetheine at the expense of its by-products: profibrotic pantothenic acid and prooxidant cysteamine/cystamine. HOCl-vnn1^{-/-}-mice display a reduction in the serum concentration of pantothenic acid that correlates with the reduction in the accumulation of collagen in skin and lung, and reduced expansion of myofibroblasts in those tissues. Furthermore, we found that HOCl-vnn1^{-/-} mice have lower levels of systemic oxidative stress reflected by the lower level of circulating AOPP and higher levels of total thiols than HOCl-WT-mice. These data are in line with previous observations that vnn1^{-/-}-mice have plethoric GSH stores and are resistant to oxidative stress-related diseases and tissue injury induced in vivo by paraquat or γ irradiation (15). As ROS play a key role in the initiation and the diffusion of fibrosis in SSc, the antioxidative effects observed in vnn1^{-/-} animal certainly also contributes to the reduced fibrosis observed in HOCl-vnn1^{-/-} mice compared to HOCl-WT-mice (3, 36, 37).

We also observed an improvement in markers of vascular dysfunction in HOCl-vnn1^{-/-} mice compared to HOCl-WT mice. The involvement of vanin-1 in this phenomenon is multiple.

First, pantethine, the substrate of vanin-1 has been associated with vascular protection in different models of inflammatory conditions including cerebral malaria and hyperlipoproteinemia (11-14). The preservation of pantethine levels secondary to the invalidation of vanin-1 could therefore contribute to the inhibition of endothelial alterations. Second, ROS are directly implicated in the vascular disease observed in SSc through a direct deleterious effect on endothelial cells (3, 5). Therefore, the invalidation of vanin-1 and the consecutive elevation in total thiols levels may also participate to the improvement of the vascular disease in HOCl-vnn1^{-/-} mice. Third, vascular dysfunction in SSc is characterized by proliferation of intimal cells and thickening of the media, and vanin-1 could also participate to that phenomenon as the enzyme has recently been described for promoting the proliferation of vascular smooth muscle cells in neo-intimal hyperplasia (38).

HOCl-vnn1^{-/-} mice also display milder signs of auto-immunity reflected by reduced levels of anti-DNA Topoisomerase-1 and reduced production of splenic Th2 cytokines. ROS play a key role in the breach of immune tolerance through the oxidation and modification of self-antigens in several auto-immune diseases including SSc (3, 26-28). In the HOCl-model of SSc, free radicals oxidize DNA Topoisomerase-1, triggering the development of a systemic Th2-mediated auto-immune response with anti-DNA Topoisomerase-1 auto-antibodies. Therefore, the reduction in the levels of systemic oxidative stress observed in HOCl-vnn1^{-/-}-mice secondary to the invalidation of the vanin-1 gene also participates to the abrogation of the auto-immune disease. Interestingly, this phenomenon doesn't seem to be limited to SSc as a recent paper reported that the Vnn1 gene and oxidative-stress related pathways are overexpressed in immune thrombocytopenia, a disorder that results from a loss of immune tolerance and the generation of auto-antibodies (39).

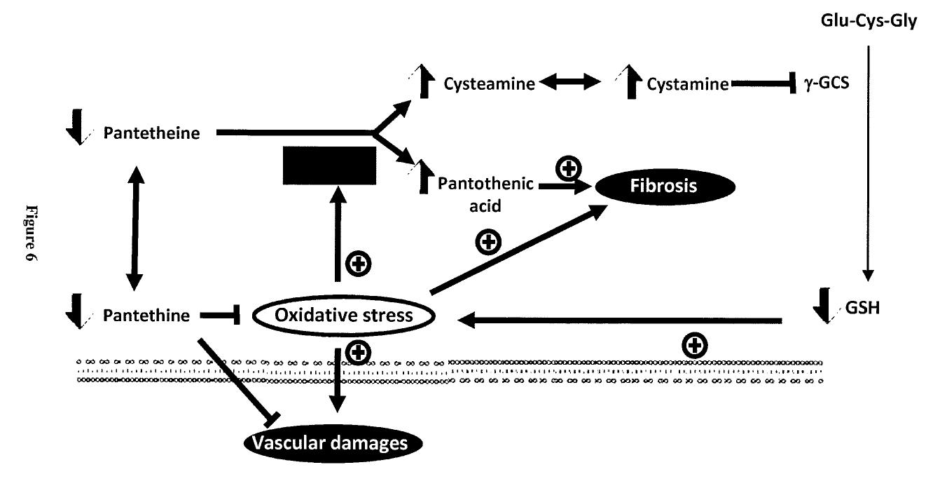

Having shown the dysregulation of the vanin-1/pantetheinase pathway in the HOCl mouse model of SSc and the beneficial impact of the invalidation of the vnn1 gene in HOCl-mice (FIG. 6), we investigated whether this observation also applies to SSc patients. As in SSc-mice, patients with SSc display a higher vanin-1 activity in skin and in serum, and higher serum pantothenic acid concentration than healthy individuals. Moreover, the highest levels of vanin-1 and pantothenic acid were observed in patients with a diffuse involvement of the disease and vascular complications. Therefore, we propose that vanin-1 activity and pantothenic acid levels could be used as biological markers of the severity of the disease in humans. These observations not only strengthen the similarities between the animal model induced by HOCl injections and the human disease, but suggest that molecules that can inhibit vanin-1 could have an anti-fibrotic, anti-inflammatory and vasculoprotective effects in SSc.

TABLE 1
Informations regarding the cohort of SSc patients of the study.
SSc patients informations

| Sex ratio | |
|---|---|
| Women/Men | 43/8 |
| Rodnan Score | |
| min | 4 |
| max | 36 |
| mean | 14 |
| Form of the disease (nb) | |
| diffuse | 22 |
| limited | 29 |
| Vascular involvement | |
| digital ulcers | 31 |
| renal crisis | 1 |
| Pulmonary arterial hypertension | 7 |
| Lung fibrosis | 17 |
| Sclerodermic cardiopathy | 5 |
| Age (years) | |
| min | 26 |
| max | 83 |
| mean | 54 |
| Auto-antibodies (nb) | |
| Anti-Nuclear Antibodies | 48 |
| Anti-CENP-B | 23 |
| Anti-Topoisomerase-1 | 13 |

### REFERENCES

Throughout this application, various references describe the state of the art to which this invention pertains. The disclosures of these references are hereby incorporated by reference into the present disclosure.
1. Gabrielli A, Avvedimento E V, Krieg T. Scleroderma. N Engl J Med 2009; 360(19):1989-2003.
2. Harris M L, Rosen A. Autoimmunity in scleroderma: the origin, pathogenetic role, and clinical significance of autoantibodies. Curr Opin Rheumatol 2003; 15(6):778-84.
3. Servettaz A, Goulvestre C, Kavian N, Nicco C, Guilpain P, Chéreau C, et al. Selective oxidation of DNA topoisomerase 1 induced systemic sclerosis in the mouse. J Immunol 2009; 182(9):5855-5864.
4. Svegliati Baroni S, Santillo M, Bevilacqua F, Luchetti M, Spadoni T, Mancini M, et al. Stimulatory autoantibodies to the PDGF receptor in systemic sclerosis. N Engl J Med 2006; 354:2667-2676.
5. Servettaz A, Guilpain P, Goulvestre C, Chereau C, Hercend C, Nicco C, et al. Radical oxygen species production induced by advanced oxidation protein products predicts clinical evolution and response to treatment in systemic sclerosis. Ann Rheum Dis. 2007; 66(9):1202-1209.
6. Kavian N, Marut W, Servettaz A, Nicco C, Chereau C, Lemarechal H, et al. Reactive oxygen species-mediated killing of activated fibroblasts by arsenic trioxide ameliorates fibrosis in a murine model of systemic sclerosis. Arthritis Rheum 2012; 64(10):3430-40.
7. Maras B, Barra D, Dupre S, Pitari G. Is pantetheinase the actual identity of mouse and human vanin-1 proteins? FEBS Lett 1999; 461(3):149-52.
8. Martin F, Malergue F, Pitari G, Philippe J M, Philips S, Chabret C, et al. Vanin genes are clustered (human 6q22-24 and mouse 10A2B1) and encode isoforms of pantetheinase ectoenzymes. Immunogenetics 2001; 53 (4):296-306.
9. Naquet P, Pitari G, Dupre S, Galland F. Role of the Vnn1 pantetheinase in tissue tolerance to stress. Biochem Soc Trans 2014; 42(4):1094-100.
10. Dupre S, Cavallini D. Purification and properties of pantetheinase from horse kidney. Methods Enzymol 1979; 62:262-7.
11. Penet M F, Abou-Hamdan M, Coltel N, Cornille E, Grau G E, de Reggi M, et al. Protection against cerebral malaria by the low-molecular-weight thiol pantethine. Proc Natl Acad Sci USA 2008; 105(4):1321-6.
12. Gaddi A, Descovich G C, Noseda G, Fragiacomo C, Colombo L, Craveri A, et al. Controlled evaluation of pantethine, a natural hypolipidemic compound, in patients with different forms of hyperlipoproteinemia. Atherosclerosis 1984; 50(1):73-83.
13. Prisco D, Rogasi P G, Matucci M, Paniccia R, Abbate R, Gensini G F, et al. Effect of oral treatment with pantethine on platelet and plasma phospholipids in IIa hyperlipoproteinemia. Angiology 1987; 38(3):241-7.
14. Eto M, Watanabe K, Chonan N, Ishii K. Lowering effect of pantethine on plasma beta-thrombo globulin and lipids in diabetes mellitus. Artery 1987; 15(1):1-12.
15. Martin F, Penet M F, Malergue F, Lepidi H, Dessein A, Galland F, et al. Vanin-1(−/−) mice show decreased NSAID- and Schistosoma-induced intestinal inflammation associated with higher glutathione stores. J Clin Invest 2004; 113(4):591-7.
16. Griffith O W, Larsson A, Meister A Inhibition of gamma-glutamylcysteine synthetase by cystamine: an approach to a therapy of 5-oxoprolinuria (pyroglutamic aciduria). Biochem Biophys Res Commun 1977; 79(3):919-25.
17. Combes V, Coltel N, Faille D, Wassmer S C, Grau G E. Cerebral malaria: role of microparticles and platelets in alterations of the blood-brain barrier. Int J Parasitol 2006; 36(5):541-6.
18. Weimann B I, Hermann D. Studies on wound healing: effects of calcium D-pantothenate on the migration, proliferation and protein synthesis of human dermal fibroblasts in culture. Int J Vitam Nutr Res 1999; 69(2):113-9.
19. Kobayashi D, Kusama M, Onda M, Nakahata N. The effect of pantothenic acid deficiency on keratinocyte proliferation and the synthesis of keratinocyte growth factor and collagen in fibroblasts. J Pharmacol Sci 2011; 115(2):230-4.
20. Jansen P A, Kamsteeg M, Rodijk-Olthuis D, van Vlijmen-Willems I M, de Jongh G J, Bergers M, et al. Expression of the vanin gene family in normal and inflamed human skin: induction by proinflammatory cytokines. J Invest Dermatol 2009; 129(9):2167-74.
21. Pitari G, Malergue F, Martin F, Philippe J M, Massucci M T, Chabret C, et al. Pantetheinase activity of membrane-bound Vanin-1: lack of free cysteamine in tissues of Vanin-1 deficient mice. FEBS Lett 2000; 483(2-3):149-54.
22. Meghari S, Berruyer C, Lepidi H, Galland F, Naquet P, Mege J L. Vanin-1 controls granuloma formation and macrophage polarization in Coxiella burnetii infection. Eur J Immunol 2007; 37(1):24-32.
23. Berruyer C, Pouyet L, Millet V, Martin F M, LeGoffic A, Canonici A, et al. Vanin-1 licenses inflammatory mediator production by gut epithelial cells and controls colitis by antagonizing peroxisome proliferator-activated receptor gamma activity. J Exp Med 2006; 203(13):2817-27.
24. Berruyer C, Martin F M, Castellano R, Macone A, Malergue F, Garrido-Urbani S, et al. Vanin-1−/− mice exhibit a glutathione-mediated tissue resistance to oxidative stress. Mol Cell Biol 2004; 24(16):7214-24.
25. Rommelaere S, Millet V, Gensollen T, Bourges C, Eeckhoute J, Hennuyer N, et al. PPARalpha regulates the production of serum Vanin-1 by liver. FEBS Lett 2013; 587(22):3742-8.
26. Kurien B T, Hensley K, Bachmann M, Scofield R H. Oxidatively modified autoantigens in autoimmune diseases. Free Radic Biol Med 2006; 41(4):549-56.
27. Griffiths H R. Is the generation of neo-antigenic determinants by free radicals central to the development of autoimmune rheumatoid disease? Autoimmun Rev 2008; 7(7):544-9.
28. Iuchi Y, Kibe N, Tsunoda S, Suzuki S, Mikami T, Okada F, et al. Implication of oxidative stress as a cause of autoimmune hemolytic anemia in NZB mice. Free Radic Biol Med 2010; 48(7):935-44.
29. Woessner J F, Jr. The determination of hydroxyproline in tissue and protein samples containing small proportions of this imino acid. Arch Biochem Biophys 1961; 93:440-7.
30. Kavian N, Servettaz A, Mongaret C, Wang A, Nicco C, Chéreau C, et al. Targeting ADAM-17/notch signaling abrogates the development of systemic sclerosis in a murine model. Arthritis Rheum 2010; 62(11):3477-3487.
31. LeRoy E C, Medsger T A J. Criteria for the classification of early systemic sclerosis. J Rheumatol 2001; 28(7):1573-6.
32. van den Hoogen F, Khanna D, Fransen J, Johnson S R, Baron M, Tyndall A, et al. Classification criteria for systemic sclerosis: an american college of rheumatology/european league against rheumatism collaborative initiative. Arthritis Rheum 2013; 65(11):2737-47.
33. Gensollen T, Bourges C, Rihet P, Rostan A, Millet V, Noguchi T, et al. Functional polymorphisms in the regulatory regions of the VNN1 gene are associated with susceptibility to inflammatory bowel diseases. Inflamm Bowel Dis 2013; 19(11):2315-25.
34. Wittwer C T, Gahl W A, Butler J D, Zatz M, Thoene J G. Metabolism of pantethine in cystinosis. J Clin Invest 1985; 76(4):1665-72.
35. Bouazza N, Treluyer J M, Ottolenghi C, Urien S, Deschenes G, Ricquier D, et al. Population pharmacokinetics and pharmacodynamics of cysteamine in nephropathic cystinosis patients. Orphanet J Rare Dis 2011; 6:86.
36. Sambo P, Baroni S S, Luchetti M, Paroncini P, Dusi S, Orlandini G, et al. Oxidative stress in scleroderma: maintenance of scleroderma fibroblast phenotype by the constitutive up-regulation of reactive oxygen species generation through the NADPH oxidase complex pathway. Arthritis Rheum 2001; 44(11):2653-64.
37. Herrick A L, Matucci Cerinic M. The emerging problem of oxidative stress and the role of antioxidants in systemic sclerosis. Clin Exp Rheumatol 2001; 19(1):4-8.
38. Dammanahalli K J, Stevens S, Terkeltaub R. Vanin-1 pantetheinase drives smooth muscle cell activation in post-arterial injury neointimal hyperplasia. PLoS One 2012; 7(6):e39106.
39. Zhang B, Lo C, Shen L, Sood R, Jones C, Cusmano-Ozog K, et al. The role of vanin-1 and oxidative stress-related pathways in distinguishing acute and chronic pediatric ITP. Blood 2011; 117(17):4569-79.
40. Kavian N, Mehlal S, Marut W, Servettaz A, Giessner C, Bourges C, Nicco C, Chéreau C, Lemaréchal H, Dutilh M F, Cerles O, Guilpain P, Vuiblet V, Chouzenoux S, Galland F, Quere I, Weill B, Naquet P, Batteux F. Imbalance of the Vanin-1 Pathway in Systemic Sclerosis. J Immunol. 2016 Oct. 15; 197(8):3326-3335.

1. A method of treating systemic sclerosis in a subject in need thereof comprising administering to the subject a therapeutically effective amount of a vanin-1 inhibitor.

2. The method of claim 1 wherein the vanin-1 inhibitor is a small organic molecule, an antibody or an inhibitor of vanin-1 expression.

3. A method of determining the severity of systemic sclerosis in a subject comprising i) determining the level of vanin-1 activity in a blood sample obtained from the subject ii) comparing the level determined at step i) with a predetermined reference value and iii) concluding that the subject suffers from a severe form of systemic sclerosis when the level determined at step i) is higher than the predetermined reference value or concluding that the subject suffers from limited form of systemic sclerosis.

4. The method of claim 3 wherein the determination of the vanin-1 activity comprises determining the vanin-1 level of and/or pantothenic acid in the blood sample.

5. A method of treating systemic sclerosis in a subject in need thereof comprising i) determining the level of vanin-1 activity in a blood sample obtained from the subject ii) comparing the level determined at step i) with a predetermined reference value and iii) administering to the subject a therapeutically effective amount of a vanin-1 inhibitor when the level determined at step i) is higher than the predetermined reference value.
